(19) United States
(12) Patent Application Publication
(10) Pub. No.: US 2002/0022060 A1
(43) Pub. Date: Feb. 21, 2002

(54) Combined plant coagulate composition, process for the manufacture thereof and uses thereof

(76) Inventors: Beena Mathur, Rajasthan (IN); Ramesh Kumar Duggal, Delhi (IN); Kottaiappan Saravanakumar, Ghaziahad (IN)

Correspondence Address: STAAS & HALSEY LLP, 700 11TH STREET, NW, SUITE 500, WASHINGTON, DC, 20001, US

(21) Appl. No.: 09/815,334

(22) Filed: Mar. 23, 2001

(30) Foreign Application Priority Data
Mar. 28, 2000 (IN) 340/DEL/2000

(51) Int. Cl. A61K 35/78

(52) U.S. Cl. 424/725; 424/757; 424/774

(57) ABSTRACT

A combined plant coagulate composition, a process for manufacturing a combined plant coagulate composition and uses thereof. The composition comprises a plant coagulate from a combination of two or more plants comprising of mainly green leaves matter, preferably among Spinach (Spinacia oleracea), Amaranth (Amaranthus spp.), Berseem (Trifolium alexandrium) and Cowpea (Vigna sinensis) for the treatment of iron deficiency related problems in both the sex and all ages. The process includes collection of juice from combined plant source, preferably expressing the juice from individual plant, mixing them and to effect coagulation in the combined juice to give plant coagulate which unexpectedly performs better in improving the blood profile than plant coagulate from individual sources.

### CROSS REFERENCE TO RELATED APPLICATION

This application is based upon and claims priority of Indian Patent Application No. 340/Del/2000 filed Mar. 28, 2000, the contents being incorporated herein by reference.

### BACKGROUND OF THE INVENTION

The present invention relates to a Combined Plant Coagulate Composition, a process for manufacturing a Combined Plant Coagulate composition and uses thereof. The composition comprises a plant coagulate from any combination of two or more plants comprising of mainly green leaves matter, preferably among Spinach (Spinacia oleracea), Amaranth (Amaranthus spp.), Berseem (Trifolium alexandranum) and Cowpea (Vigna sinensis) for the treatment of iron deficiency related problems in both the sex and all ages. The process includes collection of juice from combined plant source, preferably expressing the juice from individual plant, mixing them and to effect coagulation in the combined juice to give plant coagulate which unexpectedly performs better in improving the blood profile than plant coagulate from individual sources.

1. Field of invention

The present invention relates to a Combined Plant Coagulate Composition and a method of preparing plant coagulate from combination of plants. More particularly, the present invention relates to collection of juice from selected plants preferably those specified plants individually and mixing them followed by effecting coagulation from the combined juice. The resulting plant coagulate (and thereby the formulations containing plant coagulate alone or in combination with other ingredients) has been envisaged to be more effective in treating iron deficiency related conditions in humans than the plant coagulate prepared from individual plants.

2. Description of the Related Art

Beddoes (1792) and Lawes (1885) suggested the possibility of making human food from inedible leaves. Ereky (1927) took out the first patent on the idea. This was the forerunner of patents by many others for making food for non-ruminants from materials that could otherwise be used as ruminant food only. The use of some equipments for the process was patented by Goodall in 1936. The use of juice from grass as a medicinal product was patented by Schnakel (1938). Many patents have been allowed since 1939 although they were all for academic interest. (Pirie. N W.,1987)

Extensive studies have been conducted worldwide on different aspects of Plant coagulate/Leaf Protein Concentrate (LPC) manufacture and its applications. The studies conducted are summarized under the headings below.

(i) Plant sources for the manufacture of Plant Coagulate/leaf protein concentrate:

Numerous plants have been studied as source for the manufacture of LPC and their advantages and drawbacks are reported with respect to various qualitative and quantitative parameters. Though various plants from different categories ranging from inedible grass to edible leafy vegetables have been investigated, most of the studies are focused on few plants such as Alfalfa (Medicago sativa), Spinach (Spinacia oleracea). Indian Standards IS 8222 (1976) recommends the manufacture of LPC from any of 19 pants.

(ii) Manufacturing process of Plant coagulate/Leaf Protein Concentrate:

The manufacture of LPC essentially involves the following steps:

a. Expression of juice from the plant leaves

b. Seperation of protein from the juice

c. Collection & drying of protein.

Different types of equipments have been studied for the processing of the leaves during the manufacture of LPC. Those equipments were studied & recommended considering various parameters like process efficiency, energy input, ease of operation, cost etc. (Pirie. N W.,1987) Fardel R G has patented (U.S. Pat. No. 4,629,122, 1986) a field going machine for the manufacture of LPC.

Extensive studies have been carried out on the isolation of protein fraction from the juice. The published literature (Hernandez et al., 1988; Huang et al, 1971: Pandey. V N et al., 1993; Bray W J et al.,1979) suggests the use of following techniques for the separation of proteins from the leaf juice:

1. Heat coagulation;

2. Precipitation of protein by acidification/alkalination;

3. Precipitation of protein by solvent;

4. Protein coagulation by fermentation;

5. Use of poly electrolytes/polyanionic flocculants/cross linking agents, (De Jong et al., U.S. Pat. No. 4,333,871,1982);

6. Centrifugation/ultra filtration; and

7. Fractional seperation of proteins (Bickoff et al U.S. Pat. No. 3,959,246, 1976; Batley Jr, W R, U.S. Pat. No. 4,130,553, 1978.).

Effect of modifications in the process of preparation on nutritional value of LPC was studied by Maliwal. B P (1983). Tao.M et al (1972) reported that more elements except iron & sodium and more carbohydrates were found in solvent precipitated LPCs than in heat coagulum. The changes in the composition of LPC on storage was studied by Maliwal B P (1981); Betschart A A et al. (1974). Shah F H (1968) used dried amla powder (Emblica officinalis) to minimise the oxidation in LPC. The effect of presence of poyphenols & tanins on the nutritional properties of LPC was studied by Maliwal. B P (1983). Plastein reaction was successfully used by Vasant A et al (1980) to modify LPC into more acceptable soluble form.

(iii) Applications of Plant coagulate/Leaf Protein Concentrate:

Being rich in protein, LPC mainly finds its application as food supplement. Several publications claim the successful use of LPC in the management of malnutrition, especially in children. (Shah et al. 1981, Mathur.B et al.) Subba Rao (1971) studied wheat flour fortified with LPC for its nutritional properties in rats. Fortification with essential aminoacids methionine & lysine improved the overall nutritional qualities of LPC (Jokl. L et al., 1984)

LPC has been reported to be useful as hypocholesterolemic agent in experimental studies on rats (Satosh. A et al 1995). Mathur.B et al (1989) has proven the usefulness of LPC in improving heamoglobin levels in children.

It is noteworthy that all these studies are centered around nutritional aspects and lack specificity in clinical conditions. In addition, all these studies were carried out on LPC from single plant source. It is also equally important that these studies are not focussed to evaluate different LPC in comparative and controlled manner.

### SUMMARY OF THE INVENTION

A main objective of the present invention is to develop a process for the manufacture of plant coagulate from combined plants source, named as combined plant coagulate (CPC).

It is a further objective of the invention to optimise the efficacy of combined plant coagulate in the management of iron deficiency related conditions like anemia by process ingenuity.

A still further objective of the invention is to use CPC alone or in combination with other additional medicaments/supplements in iron deficiency related problems.

### DETAILED DESCRIPTION OF THE PREFERRED EMBODIMENTS

This invention mainly focuses on the manufacture of plant coagulate from combined plants source. It is also envisaged that the Plant Coagulate from the combined plant source is more effective in treating iron deficiency related conditions.

The plant materials combination to be used for the manufacture of CPC is first selected. In this invention, four plants Spinach (Spinacia oleracea), Amaranth (Amaranthus spp.), Berseem (Trifolium alxandranum) and Cowpea (Vignia sinensis) are taken for study as illustrative candidates.

Any two or more of the above mentioned plants can be used for the manufacture of combined plant coagulate. The selected plants are harvested at the suitable stage of growth for optimum protein concentration. Then the plants are taken in suitable proportion ranging from 10 to 90% weight/weight. The plant leaves are washed in water, if necessary in water containing sodium bisulphite to minimise the oxidation during processing.

### Expression of Juice

The plants are then pulped in suitable machine, pulper/grinder/expellar and the resultant mass is expressed by suitable equipment like Hydraulic/Pneumatic Press to collect the juice. The juice from the selected plants are collected separately.

Alternatively, the weighed quantity of selected plants may be mixed and then subjected to grinding & expression to yield the juice. However, collection of the juice from individual plant is preferred.

### Separation of Proteins

The collected juice from the selected plants are mixed and the pH of the mixed juice is checked (2.5-10.0). Then the separation of protein is effected by suitable method preferably by heating the juice employing suitable means to 40-100° C.

### Collection & Drying

Once the coagulation is complete, the coagulate are collected by filtration/ centrifugation and it is pressed by suitable equipment to dehydrate the protein to the maximum possible extent. The dehydrated mass is dried in a suitable equipment like oven/fluidised bed dryer at a temperature below 65° C. under atmospheric/reduced pressure till the moisture content is brought down below 8% as quantified by Karl Fischer Reagent. Finally the dried mass is ground into suitable particle size as desired.

Alternatively, spray drying technique may be employed to effect drying of the coagulate, under suitable process controls.

The CPC thus prepared has been studied for its usefulness in iron deficiency related conditions (anemia) in rats. Interestingly, the CPC performed better in improving the blood profile than the plant coagulate prepared from single plant source.

Animal studies have also been performed to study the difference in the efficacy among the CPC prepared from different combination of plants. Though the performance of plant coagulate from combinations is found to be better than that of single plant, no conclusive remarks could be made on the activity among the combinations of different plants.

The application of CPC in iron deficiency related conditions has also been confirmed in human subjects. The therapeutic activity of CPC has been found to be comparable to iron+folic acid tablets in a study conducted in human volunteers. This comparable efficacy with CPC comes with an additional advantage of free from gastrointestinal disturbances, often observed with iron salts therapy.

The mixture of CPC & Amla (Indian Goosberry—Emblica officinalis) was investigated for its therapeutic activity in experimental anemia in rats. The dried Amla fruit was powdered and added with CPC upto the concentration of 50%. This combination of CPC & Amla produced better results in improving the blood profile in anemic rats. It is envisaged from this experiment that herbal ingredients like amla (Emblica officinalis), Triphala (mixture of equal quantities of Emblica officinalis, Terminalia chebula & Terminalia belerica), Punarnava (Boerrhavia diffusa) and others like Lauh Bhasma, Yashad Bhasma (metallic drugs used in Ayurvedic system of medicine) may also improve the therapeutic properties of CPC.

Here it is noteworthy that an anemic person (adult) needs 50 mg of elemental iron (approx.) to be absorbed into the body and it is prescribed, in practice, in the form of iron preparations containing 60 mg of elemental iron in 3-4 doses. Interestingly, CPC, despite its very less iron content, manages the iron deficiency related conditions. Hence, it is conceptualised that CPC favours the absorption of dietary iron by some unknown mechanism(s). The Combined Plant Coagulate Composition exhibits synergistic and surprising properties.

It is also assumed that CPC has better absorption characteristics to the Plant Coagulate prepared from individual plants. It may also be possible that some components that interfere with the dietary iron absorption like saponins are not/least precipitated during the coagulation of the combined juice. The exact mechanism for this phenomena is unknown.

The invention will now be described with reference to the accompanying examples which should not be construed to limit the scope of the invention.

### EXAMPLE 1

### Preparation of Combined Plant Coagulate

In this example, Spinach and Berseem were taken for the preparation of Combined Plant Coagulate. The aerial parts of Spinach and Berseem was collected from the field in the morning. Both the plants were taken in equal quantities (2 Kg each). The plant was washed with water to remove all the adhering soil matter. Then the plants were ground using chopper mill and the ground mass was collected separately. The ground mass was then manually expressed to collect the juice. (Volume collected: Spinach-1140 ml; Berseem-980 ml) The juice collected were mixed (pH of the juice mix -6.4) and heated in an electrically heated pan to 85° C. for about 10 minutes. Then the coagulated mass was collected by filtration through standard mesh no:100 and washed with water. After washing, the coagulate was expressed to remove superficial moisture. And finally the mass was dried in hot air oven at 55° C.

This same process was followed for the preparation of all the Plant Coagulate samples, which were used in chemical/experimental/clinical studies in the following examples.

### EXAMPLE 2

Plant Coagulate from individual plant source (4 plants mentioned in the description of invention) and from combined plant source were prepared by the method as described in example-1 and were analysed for their iron content. The iron content of these samples are tabulated below:

| PC source | Iron content (% w/w) |
|---|---|
| Spinach | 0.21 |
| Cowpea | 0.33 |
| Amaranth | 0.22 |
| Berseem | 0.28 |
| Combination I | 0.25 |
| (Spinach & Barseem 25:75) | |
| Combination II | 0.26 |
| (Cowpea & Amaranth 25:75) | |

Since there is no remarkable difference in the iron content among the PC samples prepared from individual & combined plant sources, the better therapeutic activity of combined PC is attributed to some other mechanisms irrespective of iron content. The Combined Plant Coagulate Composition exhibits synergistic and surprising properties.

### EXAMPLE 3

PC from individual plant and from combined plant source (prepared by the method as mentioned in example-1) were studied for their therapeutic activity in experimental anemia in albino rats. Initially, 35 healthy animals of both the sex and suitable body weight were taken and the anemia was induced by the administration of iron-free feed (milk) for 21 days along with the withdrawal of 0.1 ml of blood every week. During this period, blood hemoglobin level fell down by about 45%. Then the experiment was conducted in 28 anemic rats divided into seven groups. PC from individual plant and from combined plant source (mentioned in this patent) were administered at the dose of 150 mg/day, to animals in different groups. During this period, the animals were fed standard diet. The blood samples were drawn from the animals periodically and their hemoglobin levels were studied.

| | | % increase in Hb * | | |
|---|---|---|---|---|
| Groups | | 1 week | 2 weeks | 3 weeks |
| I. | Control Gr. | 4.4 | 12.4 | 21.9 |
| II. | Spinach Gr. | 18.9 | 26.2 | 32.1 |
| III. | Amaranth Gr. | 30.0 | 38.2 | 41.9 |
| IV. | Cowpea Gr. | 24.2 | 30.8 | 34.4 |
| V. | Barseem Gr. | 24.8 | 31.5 | 36.9 |
| VI. | Combination I | 37.1 | 50.3 | 57.8 |
| | (Spinach & Barseem) | | | |
| VII. | Combination II | 39.4 | 52.1 | 59.2 |
| | (Cowpea & Amaranth) | | | |

* Values are average of 4 observations

It is observed from the results that PC from the combined plant source (groups VI & VII) surprisingly performs better in the treatment of anemia than PC from the individual plants, suggesting the superiority of PC from combined plant source.

### EXAMPLE 4

In a study conducted in rats, the efficacy of three samples:

1. PC prepared from single plant source(Berseem)

2. CPC prepared from the combination of plants (Berseem & Spinach 75:25) and

3. CPC with Amla (Emblica officinalis) (20:1) were evaluated in experimental anemia at different dose levels. Initially the anemia was induced in rats by the administration of iron free diet (milk) for 40 days along with the withdrawal of 0.1 ml of blood every week. Then the animals were divided into 4 groups of 8 animals each and were administered the drugs at the calculated dose for 30 days. The dose was calculated on the basis of the human dose decided. During the study, all the animals were kept on standard diet to ensure uniform intake of iron from the food. The blood samples were drawn periodically (every 10 days) from the animals and studied for various parameters and the results are tabulated below;

| | | PC | | |
|---|---|---|---|---|
| | | Group | | CPC + Amla |
| | | (Dose: | CPC Group | Group |
| | | 150 mg/ | (Dose: | (Dose: |
| Parameters | Control | day) | 36 mg/day) | 36 mg/day) |
| Initial | | | | |
| Haemoglobin (g/dl) | 9.60 | 9.87 | 9.40 | 9.90 |
| Total RBC (mil/sq. mm) | 6.28 | 6.43 | 6.21 | 6.30 |
| Haematocrit/PCV (%) | 34.00 | 35.45 | 33.70 | 35.40 |
| MCV (cub. μ) | 54.88 | 54.50 | 54.16 | 56.13 |
| MCH (%) | 15.30 | 15.40 | 15.30 | 15.80 |
| MCHC (%) | 28.00 | 27.50 | 28.50 | 27.40 |
| After 10 days | | | | |
| Haemoglobin (g/dl) | 8.35 | 10.70 | 11.10 | 11.40 |
| Total RBC (mil/sq. mm) | 4.67 | 7.26 | 7.74 | 7.24 |
| Haematocrit/PCV (%) | 28.66 | 40.40 | 41.25 | 41.42 |
| MCV (cub. μ) | 54.31 | 56.30 | 53.20 | 56.40 |
| MCH (%) | 15.02 | 14.90 | 14.80 | 15.50 |
| MCHC (%) | 27.41 | 26.50 | 27.70 | 27.80 |
| After 20 days | | | | |
| Haemoglobin (g/dl) | 9.04 | 11.10 | 11.00 | 10.90 |
| Total RBC (mil/sq. mm) | 5.72 | 7.52 | 7.10 | 6.90 |
| Haematocrit/PCV (%) | 31.49 | 41.80 | 39.10 | 42.10 |
| MCV (cub. μ) | 54.20 | 56.00 | 54.70 | 57.15 |
| MCH (%) | 14.93 | 15.28 | 15.40 | 15.80 |
| MCHC (%) | 27.41 | 26.75 | 28.60 | 27.50 |
| After 30 days | | | | |
| Haemoglobin (g/dl) | 8.97 | 11.50 | 11.80 | 11.70 |
| Total RBC (mil/sq. mm) | 5.88 | 7.61 | 7.94 | 7.90 |
| Haematocrit/PCV (%) | 30.87 | 41.52 | 42.70 | 45.12 |
| MCV (cub. μ) | 54.46 | 56.40 | 49.40 | 57.50 |
| MCH (%) | 14.73 | 16.16 | 15.16 | 16.52 |
| MCHC (%) | 27.90 | 28.10 | 27.95 | 28.30 |

* Values are average of 7-8 observations (due to the death of animals during the study)

The results indicate that:

1. CPC administered at the dose of 36 mg/day had produced results comparable to PC alone administered at 150 mg/day dose level. This implies that the high therapeutic dose of the PC can be minimised by the use of CPC; in other words, the CPC offers an advantage of effective in moderate dose which can be managed in pharmaceutical formulations; and

2. CPC +Amla (20:1) administered at the dose of 36 mg/day had produced results superior to other two groups; PC alone at 150 mg/day dose level & CPC alone administered at the dose of 36 mg/day. This implies that the addition of ingredients like Amla with CPC improve the therapeutic properties.

As combining CPC with Amla improves therapeutic qualities, it is envisaged that addition of herbal ingredients like Long Pepper (Piper longum), Triphala (mixture of equal quantities of Emblica officinalis, Terminalia chebula & Terminalia belerica) and others like Lauh Bhasma, Yashad Bhasma (drugs used in Ayurvedic medicine), spirulina, honey etc. may also influence the therapeutic activity of CPC.

### EXAMPLE 5

PC from individual plant and from combined plant source (mentioned in this patent) were studied for their activity in human volunteers suffering from anemia. The nine anemic patients having blood hemoglobin in the range of 8.0-8.5 were selected for the study. They were divided into three groups and given PC from individual sources (Spinach and Berseem) and PC from the combination of Spinach & Barseem at the dose of 10 G/day for a period of 4 weeks. During the study, all the patients were fed standard diet to maintain uniformity in their iron intake. The blood hemoglobin was measured fortnightly and the results are as below:

| | | Hb g/dl * | | |
|---|---|---|---|---|
| PCGroups | | 0 week | 2 weeks | 4 weeks |
| I. | Spinach Gr. | 8.32 | 10.02 | 11.42 |
| II. | Barseem Gr. | 8.44 | 10.31 | 12.12 |
| III. | Combination Gr. | 8.14 | 11.11 | 13.06 |
| | (Spinach & Barseem) | | | |

* Values are average of 3 observations

It is observed from the results that though all the samples of PC improved blood hemoglobin level in anemic patients, the PC from combined plant source performs better than the PC from individual source.

### EXAMPLE 6

A comparative study on the impact of CPC (prepared from the combination of Berseem & Spinach, 75:25 by a method as explained in example-i) and Iron+Folic acid tablet supplementation on hemoglobin levels and blood profile of human volunteers was conducted. The experiment was conducted in 10 healthy female college students divided into 3 groups. A daily dose of 10 G of CPC was administered for the subjects in CPC group and another group received Iron+Folic acid tablets (100 mg elemental iron & 500 μg of folic acid/day) for a period of 13 weeks. The blood samples were collected after 13 weeks and analysed for various blood parameters.

| | % increase in parameters | | |
|---|---|---|---|
| Parameters | Control | CPC group | IFA group |
| Haemoglobin (g/dl) | 0.44 | 13.27 | 5.70 |
| Haematocrit/PCV (%) | 0.32 | 16.61 | 7.80 |
| Total RBC (mil/sq. mm) | −0.27 | 11.05 | 5.22 |
| Serum iron (μg/dl) | 0.58 | 15.49 | 10.58 |
| Transferrin saturation | −0.11 | 23.98 | 12.89 |

* Values are average of 3 observations

During the study, it was observed that the patients receiving Iron+Folic acid tablets experienced adverse effects like nausea, diarrhea, constipation etc. while the group receiving CPC experienced no gastrointestinal disturbance. It is infered from the results that CPC could be a better agent for the treatment of iron deficiency related conditions than the conventional iron preparations, in particular in the aspect of adverse effects.

### EXAMPLE 7

In a clinical study, 19 anemic patients were selected and treated with CPC prepared from the combined plant source (Berseem & Spinach 25:75 prepared by a method as mentioned in example-1) for three months. CPC was administered in the form of hard gelatin capsules containing 625 mg of active ingredients at the dose of two capsules twice a day. The periodical analysis of various biochemical parameters was done and the results showed significant improvement in the blood profile.

| S. No. | Parameters | 0 day | 30^{th} day | 90^{th} day |
|---|---|---|---|---|
| 1. | Serum iron (mcg/dl) | 57.92 | 70.37 | 75.09 |
| 2. | TIBC (mcg/dl) | 388.31 | 372.90 | 345.81 |
| 3. | PCV (%) | 34 | 36.79 | 37.14 |

The blood hemoglobin level showed significant increase (from 9.39 g/dl to 14.96 g/dl) in just within one week after starting the treatment with CPC and the levels were maintained even in the post-treatment period.

Since many apparently different embodiments of the present invention could be made without departing from the spirit and scope thereof, it is intended that the description of the invention herein be interpreted as being illustrative only and not limiting in any manner whatsoever.

What is claimed is:

1. A combined plant coagulate composition having therapeutic properties comprising protein coagulate of green leafy matters from at least two herbs selected from the group comprising Spinach (Spinacia oleracea), Amaranth (Amaranthus spp.), Berseem (Trifolium alexandranum) and Cowpea (Vigna sinensis).

2. A composition as claimed in claim 1, wherein the said herbs in the composition are in a proportion in the range from 10 to 90%.

3. A composition as claimed in claim 1 or 2, which comprises other ingredients for the treatment of iron deficiency related conditions.

4. A composition as claimed in claim 3, wherein the said ingredients are food supplements.

5. A composition as claimed in claim 3, wherein the said ingredients are medicaments.

6. A composition as claimed in claim 5, wherein the medicaments are Allopathic medicine or other alternative systems of medicine.

7. A composition as claimed in claim 6, wherein the medicament may be herbal ingredient or plant derived material, preferably ingredients such as like Punarnava (Boerrhavia diffuse), Amla (Emblica officinalis), Triphala (mixture of equal quantities of Emblica officinalis, Terminalia chebula & Terminalia belerica).

8. A process for the manufacture of a plant coagulate composition having therapeutic properties which comprises:
harvesting the plants at suitable period of time;
washing the plant in water, or in an antioxidant solution;
expressing of juice from the plant individually and mixing them;
separating of proteins from the combined juice;
collecting of separated proteins; and
dehydrating & drying the said protein mass by suitable means to produce the combined plant coagulate.

9. A process as claimed in claim 8, wherein the separation of the proteins from the juice is done by suitable method, preferably heat coagulation, at a temperature 40-100° C., at the pH range of 2.5-10.

10. The combined plant coagulate composition as claimed in claim 1 for use in iron deficiency related conditions such as anemia.

11. A process for the manufacture of a combined plant coagulate composition as claimed in claim 8 or 9, which further comprises ingredients for the treatment of iron deficiency related conditions.

12. A process as claimed in claim 11, wherein the said ingredient could be a food supplements or medicaments.

13. A process as claimed in claim 12, wherein the medicament is an Allopathic medicine or other alternative systems of medicine.

14. A process as claimed in claim 13, wherein the medicament may be herbal ingredient or plant derived material, such as Punarnava (Boerrhavia difusa), Amla (Emblica officinalis), Triphala (mixture of equal quantities of Emblica officinalis, Terminalia chebula & Terminalia belerica).
